(19) United States
(12) Patent Application Publication
(10) Pub. No.: US 2002/0042379 A1
(43) Pub. Date: Apr. 11, 2002

(54) Administration of an anti-endotoxin drug by bolus or intermittent intravenous infusion

(76) Inventors: Daniel Rossignol, Mahwah, NJ (US); Melvyn Lynn, East Brunswick, NJ (US); Clifford DiLea, Spring Lake, NJ (US)

Correspondence Address: CLARK & ELBING LLP, 176 FEDERAL STREET, BOSTON, MA, 02110-2214, US

(21) Appl. No.: 09/879,718

(22) Filed: Jun. 11, 2001

Related U.S. Application Data
(60) Provisional application No. 60/210,638, filed on Jun. 9, 2000.

(51) Int. Cl. A61K 31/70

(52) U.S. Cl. 514/23

(57) ABSTRACT

The invention provides methods for administering an anti-endotoxin drug, E5564, by bolus or intermittent intravenous infusion. The methods can be used for treating conditions such as endotoxemia, sepsis, and septic shock.

### PRIORITY INFORMATION

This application claims the benefit of the filing date of U.S. Provisional Patent Application Serial No. 60/210,638, filed on Jun. 9, 2000, which is incorporated herein by reference.

### FIELD OF THE INVENTION

This invention relates to a regimen of administration of an anti-endotoxin drug.

### BACKGROUND OF THE INVENTION

Since the 1930's, the increasing use of immunosuppressive therapy and invasive devices, as well as the increased incidence of antibiotic resistance in bacteria, have led to a gradual rise in the occurrence of sepsis and septic shock. Currently, the estimated incidences in the U.S. of sepsis and septic shock are greater than 400,000 and greater than 200,000 patients/year, respectively. This results in about 100,000 fatalities/year, making septic shock the most common non-coronary cause of death in the hospital Intensive Care Unit (ICU). Currently, ICU therapy for septic shock is limited to antibiotic therapy, cardiovascular resuscitation, vasopressor/ionotrope therapy, and ventilatory support. This ICU care can cost up to $1,500/day and average a total of $13,000 to $30,000 per patient. Clearly, any therapy that can reduce the morbidity and mortality, and therefore the cost of care, relating to sepsis/septic shock will be of great value.

It is likely that antibiotics themselves can worsen morbidity associated with sepsis; their bactericidal action can result in the release of endotoxin from Gram negative bacteria, which are believed to induce many pathophysiological events such as fever, shock, disseminated intravascular coagulation (DIC), and hypotension. Consequently, medicines for the treatment of Gram negative sepsis have been desired for some time, especially drugs capable of blocking endotoxin or cytokines derived from endotoxin-mediated cellular stimulation. To this end, various strategies for treatment have included administration of antibodies or other agents against LPS or cytokines, such as TNF-α and interleukin-1. For various reasons, these approaches have failed.

While endotoxin itself is a highly heterogenous molecule, the expression of many of the toxic properties of endotoxin is attributed to a highly conserved hydrophobic lipid A portion. An effective drug that acts as an antagonist to this conserved structure is known as E5564 (also known as compound 1287 and SGEA). This drug is described as compound 1 in U.S. Pat. No. 5,681,824, which is hereby incorporated by reference. E5564 has the formula:

(α-D-Glucopyranose, 3-O-decyl-2-deoxy-6-O-[2-deoxy-3-O-[(3R)-3-methoxydecyl)-6-O-methyl-2-[[(11Z)-1-oxo-11-octadecenyl)amino]-4-O-phosphono-β-D-glucopyranosyl]-2-[(1,3-dioxotetradecyl)amino]-,1-(dihydrogen phosphate),

which can be provided as a tetrasodium salt. E5564 has a molecular weight of 1401.6.

### SUMMARY OF THE INVENTION

We have discovered that administration of E5564 by loading dose followed by maintenance dosing, or by single bolus injection, is effective in the prevention or treatment of the clinical effects of endotoxemia.

Accordingly, the invention features a method of treating a human patient that has or is at risk of developing a medical condition that is amenable to treatment with Compound E5564. In this method, Compound E5564 is administered to the patient by bolus or intermittent intravenous infusion. The bolus infusion can be of 0.4-60 mg, e.g., 6-56 mg or 2-28 mg drug, over the course of, e.g., 4 hours. The administration can be by intermittent infusion, in which a loading dose (of, e.g., 0.4-60 mg, 6-56 mg, or 12-28 mg drug, over a period of, e.g., 4 hours) is administered, followed by a maintenance dose. Optionally, a second loading dose (of, e.g., 0.4-60 mg, 6-56 mg, or 12-28 mg drug, over a period of, e.g., 2 hours) can be administered at about 12 or about 24 hours after the first loading dose. The maintenance dose can be administered over a period of, e.g., 2 hours, about 12 hours after the previous loading dose. Also, an additional maintenance dose, or additional maintenance doses, can be administered, that are each administered over a period of 2 hours, about 12 hours from the previous maintenance dose.

In a specific example of a method of the invention, a first loading dose of 3 mg/hour is administered for four hours, followed by a second loading dose of 3 mg/hour for two hours at 12 hours after the first loading dose, followed by a maintenance dose of 1.5 mg/hour for two hours at 12, 24, 36, 48, 60, 72, 84, 96, and 108 hours after the second loading dose.

Patients that can be treated according to the methods of the invention include, for example, surgical patients (e.g., cardiac surgical patients), patients that have or are at risk of developing endotoxemia, sepsis, or septic shock, patients that are infected with HIV, and patients that are suffering from an immunological disorder, such as graft-versus-host disease and allograft rejection.

The invention also includes the use of E5564, in the dosages set forth above, in the treatment of the conditions set forth above, as well as the use of E5564, in the dosages set forth above, in the preparation of medicaments for treating these conditions.

The methods of the invention provide significant therapeutic benefits, and are easily carried out, especially with many of the patients treated according to the methods of the invention, who already have intravenous lines inserted, as part of their treatment in the ICU. Other features and advantages of the invention will be apparent from the following detailed description and the claims.

### DETAILED DESCRIPTION

As is noted above, we have discovered that administration of E5564 to humans in a single bolus or by intermittent infusion is effective at preventing the clinical effects of endotoxemia. Our previous studies have shown that the activity of the drug decreases rapidly upon cessation of administration, although it is not cleared from circulation, indicating the desirability of continuous infusion. We have now discovered that administration of doses of the drug that are higher than the minimum dose found to be effective in endotoxin challenge studies results in prolonged activity of the drug, after cessation of administration. Thus, the present invention relates to drug administration regimens involving single bolus or intermittent infusion, to prevent or treat endotoxemia and related conditions and disorders (e.g., sepsis) in humans.

According to the invention, the drug can be administered in a single bolus by intravenous infusion through, for example, a central access line or a peripheral venous line, or by direct injection, using a syringe. Such administration may be desirable if a patient is only at short-term risk for exposure to endotoxin, and thus does not need prolonged persistence of the drug. For example, this mode of administration may be desirable in surgical patients, such as patients having cardiac surgery, e.g., coronary artery bypass graft surgery or valve replacement surgery. In these patients, a single bolus infusion of, e.g., 0.10-15 mg/hour (e.g., 0.5-7 mg/hour, or 3 mg/hour), can be administered over a period of four hours prior to and/or during surgery. (Note that the amount of drug administered is based on an assumed average weight of a patient of 70 kg.) Shorter or longer time periods of administration may be used, as determined to be appropriate by one of skill in this art, provided that the absolute amount of drug administered, as indicated above, is maintained.

In cases in which longer term persistence of active drug is desirable, for example, in the treatment of a condition associated with long-term exposure to endotoxin, such as during infection, sepsis, when a patient is immunocompromised, or in surgical situations in which it is determined that prolonged treatment is desirable, intermittent administration can be carried out. In these methods, a loading dose is administered, followed by either (i) a second loading dose and a maintenance dose (or doses), or (ii) a maintenance dose or doses, without a second loading dose, as determined to be appropriate by one of skill in this art.

The first (or only) loading dose can be administered in a manner similar to that described for the single bolus infusion described above. That is, 0.10-15 mg/hour (e.g., 0.5-7 mg/hour or 3 mg/hour), can be administered to a patient over a period of four hours prior to surgery. (As is noted above, and is applicable throughout this description, the time periods of administration can be varied, provided that dosage levels are maintained.) If a second loading dosage is to be used, it can be administered about 12 hours after the initial loading dose, and can involve infusion of, e.g., 0.10-15 mg/hour (e.g., 0.5-7 mg/hour or 3 mg/hour) of drug over a period of, e.g., about two hours.

To extend the activity of the drug, a maintenance dose (or doses) of drug can be administered, so that levels of active drug are maintained in the blood of a patient. Maintenance doses can be administered at levels that are less than the loading dose(s), for example, at a level that is about ⅙ of the loading dose. Specific amounts to be administered in maintenance doses can be determined by a medical professional, with the goal that drug level is at least maintained. Maintenance doses can be administered, for example, for about 2 hours every 12 hours beginning at hour 24 and continuing at, for example, hours 36, 48, 60, 72, 84, 96, 108, and 120. Of course, maintenance doses can be stopped at any point during this time frame, as determined to be appropriate by a medical professional.

Specific examples of dosing regimens that are included in the invention are shown in the following table.

TABLE 1
dose levels and rates of E5564 administration to
provide protection for 6 days

| | Loading Doses | | | | | |
|---|---|---|---|---|---|---|
| | Loading | Loading | Maintenance Dose³ | | | |
| | dose #1 | dose #1 | | Rate | | Total |
| Dose | (mg/hour) | (mg/hour) | Dose | (mg/hour) | Dose | Dose |
| level | for 4 hours¹ | for 2 hours² | (mg) | for 2 hours | (mg) | (mg) |
| I | 1 | 1 | 6 | 0.5 | 1 | 15 |
| II | 3 | 3 | 18 | 1.5 | 3 | 45 |
| III | 7 | 7 | 42 | 3.5 | 7 | 105 |

¹Loading dose #1 given over 4 hours at beginning of treatment only.
²Loading dose #2 given over 2 hours at 12 hours only.
³Maintenance doses given over 2 hours every 12 hours at hours 24, 36, 48, 60, 72, 84, 96, 108, and 120, for a total of 9 maintenance doses.

The methods of the invention can be used in conjunction with any type of surgery or medical procedure that could lead to the occurrence of endotoxemia or related complications (e.g., sepsis syndrome). For example, the methods of the invention can be used in conjunction with cardiac surgery (e.g., coronary artery bypass graft, cardiopulmonary bypass, or valve replacement), transplantation (of, e.g., liver, heart, kidney, or bone marrow), cancer surgery (e.g., removal of a tumor), or any abdominal surgery. Additional examples of surgical procedures with which the methods of the invention can be used are surgery for treating acute pancreatitis, inflammatory bowel disease, placement of a transjugular intrahepatic portosystemic stent shunt, hepatic resection, bum wound revision, and burn wound escharectomy. The methods of the invention can also be used in conjunction with non-surgical procedures in which the gastrointestinal tract is compromised. For example, the methods of the invention can be used in association with chemotherapy or radiation therapy in the treatment of cancer. The methods can also be used in the treatment of conditions associated with HIV infection, and immunological disorders, such as graft-versus-host disease and allograft rejection. Compound E5564 is described in U.S. Pat. No. 5,935,938, which is hereby incorporated by reference. The drug can be formulated, for example, by dissolving 35.4 mg of drug substance in 52.1 ml 0.01N NaOH, stirring for one hour at room temperature, and diluting into phosphate-buffered lactose. After adjusting the pH to 7.3 and diluting to a final concentration of 0.1 mg/ml E5564, the solution can be filter-sterilized and lyophilized. An example of a formulation of drug product in 1 ml vials is shown below.

TABLE 2

| | |
|---|---|
| Material | amount |

| | | |
|---|---|---|
| E5564 | 100 | μg |
| NaH₂PO₄.4H₂O | 0.35 | mg |
| NaOH | 0.06 | mg |
| Lactose hydrous | 100 | mg |
| Na₂HPO₄.7H₂O | 0.45 | mg |
| sterile water | 1 | ml |

As is noted above, the drug is administered by infusion, either through a central access line or a peripheral venous line, or by direct infusion by use of a syringe.

What is claimed is:

1. A method of treating a human patient that has or is at risk of developing a medical condition that is amenable to treatment with Compound E5564, said method comprising administering Compound E5564 to said patient by bolus or intermittent intravenous infusion.

2. The method of claim 1, wherein said administration is by bolus infusion of 0.4-60 mg drug.

3. The method of claim 2, wherein said administration is by bolus infusion of 6-56 mg drug.

4. The method of claim 3, wherein said administration is by bolus infusion of 12-28 mg drug.

5. The method of claim 2, wherein said administration is over the course of 4 hours.

6. The method of claim 1, wherein said administration is by intermittent infusion, comprising administration of a loading dose, followed by a maintenance dose.

7. The method of claim 6, wherein said loading dose is 0.4-60 mg, 6-56 mg, or 12-28 mg drug.

8. The method of claim 6, wherein said loading dose is over a period of 4 hours.

9. The method of claim 6, further comprising administration of a second loading dose at 12 hours after the first loading dose.

10. The method of claim 6, further comprising administration of a second loading dose at 24 hours after the first loading dose.

11. The method of claim 10, wherein the second loading dose is 0.4-60 mg, 6-56 mg, or 12-28 mg drug, administered over a period of 2 hours.

12. The method of claim 6, wherein the maintenance dose is administered over a period of 2 hours, 12 hours after the previous loading dose.

13. The method of claim 12, further comprising administration of an additional maintenance dose or additional maintenance doses, that are each administered over a period of 2 hours, 12 hours from the previous maintenance dose.

14. The method of claim 6, wherein a first loading dose of 3 mg/hour is administered for four hours, followed by a second loading dose of 3 mg/hour for two hours at 12 hours after the first loading dose, followed by a maintenance dose of 1.5 mg/hour for two hours at 12, 24, 36, 48, 60, 72, 84, 96, and 108 hours after the second loading dose.

15. The method of claim 1, wherein said patient is a surgical patient.

16. The method of claim 15, wherein said surgical patient is a cardiac surgical patient.

17. The method of claim 1, wherein the patient has or is at risk of developing endotoxemia, sepsis, or septic shock.

18. The method of claim 1, wherein the patient is infected with HIV.

19. The method of claim 1, wherein the patient is suffering from an immunological disorder.
